(19) United States
(12) Patent Application Publication
(10) Pub. No.: US 2026/0141999 A1
(43) Pub. Date: May 21, 2026

(54) INFORMATION PROCESSING METHOD, INFORMATION PROCESSING SYSTEM, AND INFORMATION PROCESSING DEVICE

(71) Applicant: Sony Group Corporation, Tokyo (JP)

(72) Inventor: Masanori Katsu, Tokyo (JP)

(73) Assignee: Sony Group Corporation, Tokyo (JP)

(21) Appl. No.: 18/704,160

(22) PCT Filed: Oct. 20, 2022
(86) PCT No.: PCT/JP2022/039082

§ 371 Date: Apr. 24, 2024

(30) Foreign Application Priority Data
Nov. 4, 2021 (JP) 2021-180078

(51) Int. Cl. G16H 10/60; G16H 20/10

(52) U.S. Cl. CPC G16H 10/60; G16H 20/10

(57) ABSTRACT

An information processing method uses a procedure in which an information processing device (1) receives sensing data of a sensor (11) provided for a user (U) being a patient, extracts a situation to be highlighted from among the user (U)'s daily situations grasped from the received sensing data, and generates content including a comment on the extracted situation. The comment includes an inquiry comment.

### FIELD

The present disclosure relates to an information processing method, an information processing system, and an information processing device.

### BACKGROUND

For example, Patent Literature 1 discloses a technology of displaying a question and an answer option, determined to be necessary according to a symptom.

### CITATION LIST

### Patent Literature

Patent Literature 1: JP 2020-57087 A
Patent Literature 2: WO 2016/185913 A
Patent Literature 3: JP 6149084 B2

### SUMMARY

### Technical Problem

It is useful to grasp daily situations of a patient at the time of diagnosis or the like. Although it is possible to grasp the situation to some extent from sensing data obtained by the sensor provided for the patient, it is not sufficient. In a medical interview performed at the time of diagnosis, it is often difficult to grasp the detailed situation because of insufficient communication ability and memory of the patient.

According to one aspect of the present disclosure, it is possible to grasp daily situations of a patient that is difficult to grasp only from sensing data.

### Solution to Problem

An information processing method according to one aspect of the present disclosure is a method to be implemented by an information processing device, the method includes: receiving sensing data obtained by a sensor provided for a user being a patient; extracting a situation to be highlighted among daily situations of the user, the situations grasped from the received sensing data; and generating content including a comment on the extracted situation, wherein the comment includes an inquiry comment.

An information processing system according to one aspect of the present disclosure includes: an information acquisition device that acquires sensing data of a sensor provided for a user being a patient; and an information processing device that extracts a situation to be highlighted from daily situations of the user, the situations grasped from the sensing data acquired by the information acquisition device, and generates content including a comment on the extracted situation, wherein the comment includes an inquiry comment.

An information processing device according to one aspect of the present disclosure includes: an extraction unit that extracts a situation to be highlighted from among daily situations of a user being a patient, the situations grasped from sensing data of a sensor provided for the user; and a generation unit that generates content including a comment on the situation extracted by the extraction unit, wherein the comment includes an inquiry comment.

### BRIEF DESCRIPTION OF DRAWINGS

FIG. 1 is a diagram illustrating an example of a schematic configuration of an information processing system 100 applicable in an information processing method according to an embodiment.

FIG. 2 is a diagram illustrating an example of a sensor 11.

FIG. 3 is a block diagram illustrating an example of a schematic configuration of an information processing system.

FIG. 4 is a diagram illustrating an example of a situation to be highlighted.

FIG. 5 is a diagram illustrating an example of comment information 253.

FIG. 6 is a diagram illustrating an example of communication.

FIG. 7 is a diagram illustrating an example of communication.

FIG. 8 is a flowchart illustrating an example of processing (information processing method) executed in the information processing system.

FIG. 9 is a diagram illustrating an example of content presentation.

FIG. 10 is a diagram illustrating an example of content presentation.

FIG. 11 is a diagram illustrating an example of content presentation.

FIG. 12 is a diagram illustrating an example of content presentation.

FIG. 13 is a diagram illustrating an example of content presentation.

FIG. 14 is a diagram illustrating an example of content presentation.

FIG. 15 is a diagram illustrating an example of content presentation.

FIG. 16 is a diagram illustrating an example of further utilization of result information and the information processing system.

FIG. 17 is a block diagram illustrating an example of a hardware configuration.

### DESCRIPTION OF EMBODIMENTS

Embodiments of the present disclosure will be described below in detail with reference to the drawings. Note that, in each of the following embodiments, the same elements are denoted by the same reference symbols, and a repetitive description thereof will be omitted.

The present disclosure will be described in the following order.
1. Embodiments
2. Modification
3. Hardware configuration example
4. Exemplary effects

### 1. Embodiments

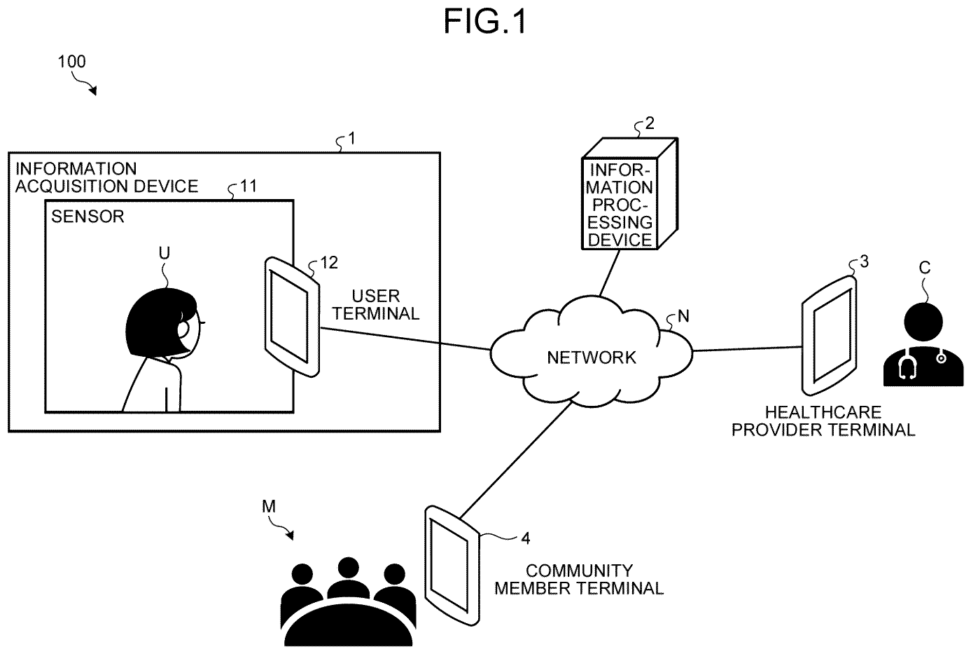

FIG. 1 is a diagram illustrating an example of a schematic configuration of an information processing system 100 applicable in an information processing method according to an embodiment. With the use of the information processing system 100, daily situations of patients are grasped. The situation includes a symptom, a physical condition, behavior, and the like of the patient, and may be construed to include a surrounding environment of the patient, such as weather. In FIG. 1, a patient is referred to as a user U. Examples of disease of the user U include heart failure, a neurological disease, a myopathy, and frailty. The user U may use a walking aid such as a wheelchair and a walker, other vehicles, or the like.

The information processing system 100 includes an information acquisition device 1, an information processing device 2, a healthcare provider terminal 3, and a community member terminal 4. These devices and terminals are configured to be able to communicate via a network N.

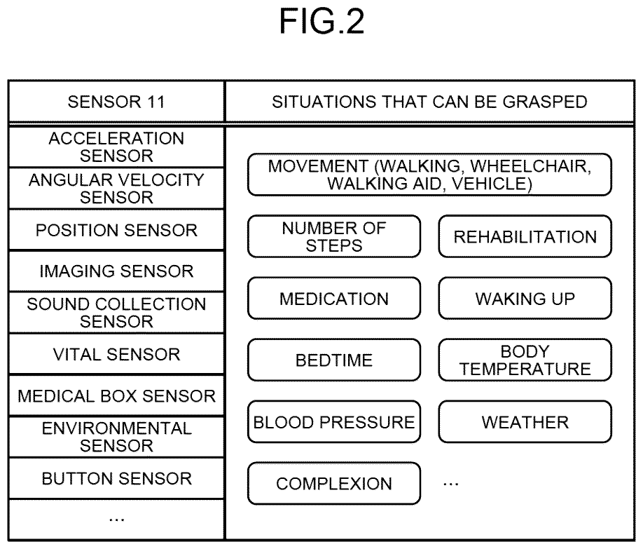

The information acquisition device 1 acquires sensing data (detection result) of a sensor provided for the user U. The sensing data is, for example, time-series data. The information acquisition device 1 includes a sensor 11 and a user terminal 12. The sensor 11 may include a wearable sensor attached to the user U. Various known sensors may be used. The sensor 11 will be described with reference to FIG. 2.

FIG. 2 is a diagram illustrating an example of the sensor 11. Examples of the sensor 11 include an acceleration sensor, an angular velocity sensor (gyro sensor), a position sensor, an imaging sensor, a sound collection sensor, a vital sensor, a medicine box sensor, an environmental sensor, and a button sensor. The acceleration sensor, the angular velocity sensor, the position sensor, the imaging sensor, and the sound collection sensor have functions indicated by their names.

The vital sensor is a sensor that detects biological information. Examples of the biological information include heart rate, brain waves, electrocardiogram, blood flow, blood pressure, respiration, lung capacity, myoelectric potential, body temperature, blood sugar level, weight, vibration, impact, and the like.

The medicine box sensor detects a state of a medicine box containing a medicine to be taken by the user U. For example, the type and amount of the medicine contained (remaining) in the medicine box are detected by a weight sensor. In other words, the type and amount of the medicine removed from the medicine box and taken by the user U are detected. In addition, for example, an illuminance sensor records when opening/closing of the medicine box is performed and detects the medication time of the user U.

The environmental sensor detects information such as temperature, humidity, and atmospheric phenomenon (weather), for example. The environmental sensor may be installed outdoors. Weather information or the like (an example of external information to be described below) available from a website of the Meteorological Agency or the like may be handled similarly to the sensing data of the environmental sensor.

The button sensor detects pressing of a button by the user U, for example. The button sensor is used to detect an occurrence of a specific situation in which the user U feels pain, for example.

The daily situation of the user U can be grasped (estimated or the like) from the sensing data of the sensor 11. FIG. 2 exemplifies, as situations that can be grasped, movement (walking, wheelchair, walking aid, vehicle), the number of steps, rehabilitation, medication, waking up, bedtime, body temperature, blood pressure, weather, and complexion. Various situations other than the illustrated situations can be grasped, and various grasping methods may be used. For example, the behavior of the user U is grasped, based on a motion, from sensing data of an acceleration sensor, an angular velocity sensor, or the like. The expression, posture, angle, and the like of the user U are grasped based on the captured image (video) from the sensing data of the imaging sensor or the like. The grasping of the angle leads to grasping of a state of the user U, such as facing down, lowering one shoulder, development of hunchback, for example. The situation of the user U is grasped, based on a sound, from the sensing data of the sound collection sensor or the like. For example, it is grasped that the user U has pain based on a voice such as “Ouch!”.

Returning to FIG. 1, the user terminal 12 is a terminal used by the user U. Examples of the user terminal 12 include a smartphone and a television. A part of the above-described sensor 11 may be installed on the user terminal 12. The user terminal 12 collects sensing data of the sensor 11 and transmits the collected sensing data to the information processing device 2. The information processing device 2 receives the sensing data. Details of the information processing device 2 will be described below.

The healthcare provider terminal 3 is a terminal used by a healthcare provider C. Examples of the healthcare provider C include doctors, nurses, physical therapists, and care workers. The community member terminal 4 is a terminal used by a member M. The member M is a member belonging to the same community as the user U, and is, for example, a family member of the user U, another patient having a disease similar to that of the user U, or the like.

The healthcare provider C will be further described. For example, the healthcare provider C being a doctor makes a diagnosis on the user U. In the diagnosis, it is also important to grasp daily situations of the user U. For example, in a case where the user U is a patient with heart failure or the like, the function decline often progresses with repeated exacerbation and remission. In a case where the user U has myopathy, frailty, or the like, a degree of progress of a symptom or physical strength are reflected in a ratio of movement means such as walking, moving using a wheelchair or a walking aid, or using a vehicle, and a sign of depression is observed in a symptom such as difficulty sleeping.

The daily situation of the user U can be grasped to some extent from the sensing data of the sensor 11 described above, but the grasping is not sufficient. For example, with the sensing data, it is possible to grasp the situation where the user U has not walked but it is difficult to grasp the situation where the user U has not walked for a certain problem or just because of having no intention of walking. Even with a medical interview performed at the time of diagnosis, the user U might have difficulty recalling the past situation or appropriately convey the past situation. For example, the problem becomes apparent in a case where the user U is a child (pediatric patient, or the like) or an elderly person. It is conceivable to perform diagnosis and medical interview with less time intervals, but this would cause strain on medical resources.

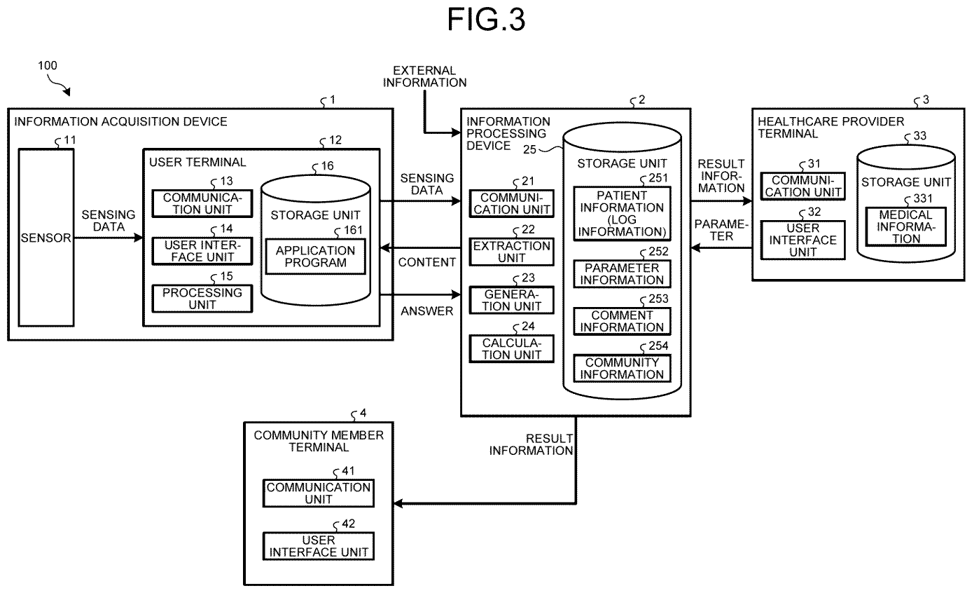

Although details will be described below, the information processing system 100 presents complementary and specific inquiry comments to the user U. From the answers of the user U, it is possible to grasp the daily situation (detailed situation) of the user U that is difficult to grasp from the sensing data. The configuration of the information processing system 100 will be further described with reference to FIG. 3.

FIG. 3 is a block diagram illustrating an example of a schematic configuration of an information processing system. As described above, in the information acquisition device 1, the sensing data from the sensor 11 is transmitted to the user terminal 12 and collected.

The user terminal 12 includes a communication unit 13, a user interface unit 14, a processing unit 15, and a storage unit 16. An example of the information stored in the storage unit 16 is an application program 161 (software) used to provide an application.

The communication unit 13 communicates with other devices and the like. For example, the communication unit 13 receives sensing data from the sensor 11 and content (described below) from the information processing device 2. Furthermore, the communication unit 13 transmits the sensing data and the answer (to be described below) of the user U to the information processing device 2.

The user interface unit 14 receives an operation of the user terminal 12 by the user U and presents information to the user U. Specific examples will be described below.

The processing unit 15 functions as a control unit that controls each element of the user terminal 12 and executes various types of processing. For example, the processing unit 15 executes the application program 161 to provide the user U with an application.

An example of the application is a rehabilitation application. For example, a rehabilitation menu indicating contents of rehabilitation or the like is presented (displayed, etc.) by the user interface unit 14. The user U performs rehabilitation according to the presented rehabilitation menu. Another example of an application is a content presenting application. Content including an inquiry comment and the like to be described below is presented by the user interface unit 14. An answer given by the user U to the presented inquiry comment is received by the user interface unit 14.

The information processing device 2 receives and processes the sensing data from the information acquisition device 1. The information processing device 2 includes a communication unit 21, an extraction unit 22, a generation unit 23, a calculation unit 24, and a storage unit 25. Examples of the information stored in the storage unit 25 include patient information (log information) 251, parameter information 252, comment information 253, and community information 254.

The patient information 251 includes information related to the user U. Examples of the patient information 251 include information related to the user U, such as disease information, hospital visit history information, rehabilitation history information, and daily state history information. The disease information includes information such as the type of disease of the user U. The hospital visit history information is information related to the past hospital visit of the user U, and is given from the outside of the information processing device 2 (such as the healthcare provider terminal 3), for example. The rehabilitation history information, which is information related to past rehabilitation applied on the user U, is acquired by a rehabilitation application executed by the user terminal 12, for example, and is transmitted from the user terminal 12 to the information processing device 2. The daily state history information is information such as a daily situation of the user U and includes sensing data. As described above, sensing data may be time-series data, that is, log information indicating the detection value of the sensor 11 for each time.

The parameter information 252 and the comment information 253 will be described below. The community information 254 is information related to a community to which the user U belongs, and includes information related to the member M and the community member terminal 4.

The communication unit 21 communicates with other devices and the like. For example, the communication unit 21 receives sensing data from the user terminal 12 and an answer given by the user U to the inquiry comment. Note that, in this example, the communication unit 21 also receives external information obtained from the outside of the information processing system 100. An example of the external information is information such as a social networking service (SNS) post from the user U, and is acquired via the Internet, for example. Another example of the external information is weather information available from the website of the Meteorological Agency or the like, and may be handled similarly to the sensing data of the environmental sensor described above. The external information is stored in the storage unit 25, for example, as a part of the patient information 251.

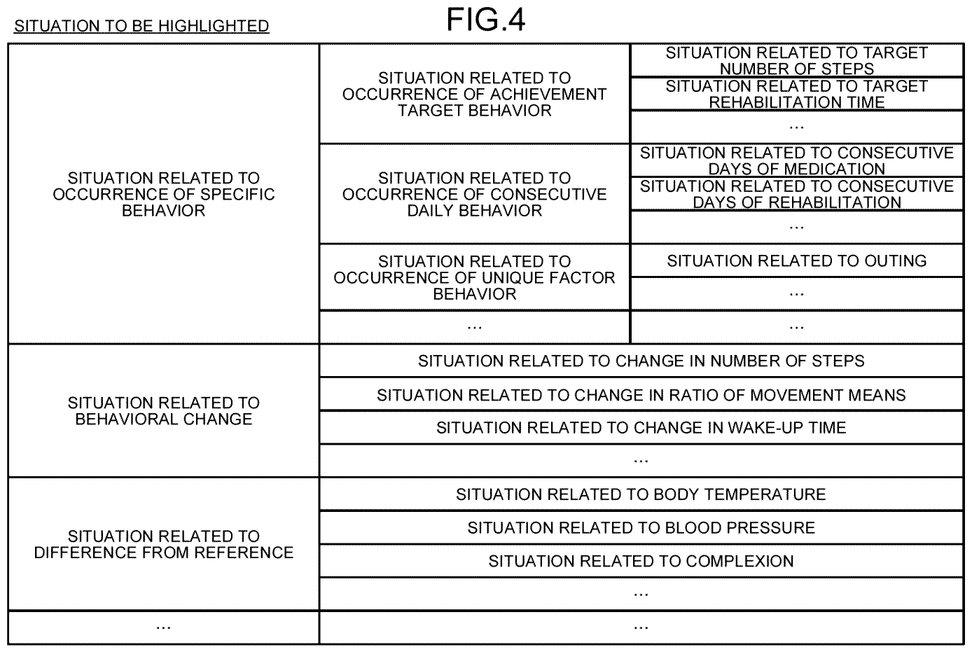

The extraction unit 22 extracts a situation to be highlighted (to be noted) from among user U's daily situations that can be grasped from the sensing data. The situation to be highlighted will be described with reference to FIG. 4.

FIG. 4 is a diagram illustrating an example of a situation to be highlighted. When the exemplified situation has been grasped from the sensing data, the extraction unit 22 extracts the situation as a situation to be highlighted. Note that grasping of the situation based on the sensing data is also performed by the extraction unit 22.

FIG. 4 illustrates, as the situations to be highlighted, “situation related to occurrence of specific behavior”, “situation related to behavioral change”, and “situation related to difference from reference”.

Examples of the specific behavior in the “situation related to occurrence of specific behavior” include an achievement target behavior in which a target to be achieved is set, a consecutive daily behavior that is consecutively performed every day, and a unique factor behavior that is set according to a symptom of the user U, a doctor's judgment, or the like. FIG. 4 illustrates, as the situation related to occurrence of a specific behavior, a situation related to occurrence of an achievement target behavior, a situation related to occurrence of a consecutive daily behavior, and a situation related to occurrence of a unique factor behavior.

The “situation related to occurrence of achievement target behavior” indicates, for example, that the target has been reached, that the target has not been reached, that the target is likely to be reached, that the target is unlikely to be reached, and the like. Examples of the achievement target behavior include the target number of steps and the target rehabilitation time. FIG. 4 illustrates a situation related to the target number of steps and a situation related to the target rehabilitation time, as the situation related to occurrence of an achievement target behavior.

The “situation related to the target number of steps” indicates that the number of steps in a predetermined period such as one day or one week has reached the target number of steps, has not reached the target number of steps, is likely to reach the target number of steps, or is unlikely to reach the target number of steps. The situation related to the target rehabilitation time indicates, for example, that the rehabilitation time has reached the target rehabilitation time, has not reached the target rehabilitation time, is likely to reach the target rehabilitation time, or is unlikely to reach the target rehabilitation time.

Determination of whether the target is likely to be reached or not is made based on a difference between the target and the current state. For example, in a case where no rehabilitation has been performed at the point of Thursday even though the rehabilitation time for two rehabilitation sessions is set as the target rehabilitation time during the period from Monday to Friday, the goal is not likely to be achieved if one more day is past with no rehabilitation. In such a case, it is determined that the target rehabilitation time is unlikely to be reached.

The “situation related to occurrence of consecutive daily behavior” indicates, for example, behavior performed for consecutive days. Examples of the consecutive daily behavior include the number of consecutive days of medication and the number of consecutive days of rehabilitation.

Examples of the “situation related to occurrence of consecutive daily behavior”, include a situation related to the number of consecutive days of medication and a situation related to the number of consecutive days of rehabilitation. The situation related to the behavior regarding the number of consecutive days of medication indicates, for example, that daily medication has been performed or has not been performed consecutively over a certain number of days (or a period of unprecedented length), such as five days or ten days. The situation related to the number of consecutive days of rehabilitation indicates, for example, that daily rehabilitation has been consecutively performed or has not been performed for a certain number of days.

An example of the “situation related to occurrence of unique factor behavior”, is a situation related to outing. The situation related to outing indicates, for example, that the outing has been performed or has not been performed, that the daily outing has been performed or has not been performed consecutively for a certain number of days. For example, setting of such a unique factor behavior is useful in a case where there is a specific relationship such as a decrease in the number of times of outing indicates a depression tendency.

The behavioral change in the “situation related to behavioral change” includes a change in the number of steps, a change in a ratio of movement means, a change in wake-up time, and the like. FIG. 4 illustrates, as the situation related to the behavioral change, a situation related to a change in the number of steps, a situation related to a change in the ratio of the movement means, and a situation related to a change in the wake-up time.

The “situation related to the change in the number of steps” indicates that the number of steps has changed, more specifically, that the tendency of the increase in the cumulative number of steps has been heightened or lowered, for example.

The “situation related to change in ratio of movement means” indicates that there is a change in the ratio of walking movement, movement using a wheelchair, use of a walking aid, movement using a vehicle, or the like with respect to movement, and more specifically, indicates that the ratio has increased or decreased, for example. The movement may be movement of a route that the user U usually uses (habitually uses). The detection of whether the route is such a route, that is, the habitual matter detection may be performed by the technique described in Patent Literature 2, for example.

The “situation related to change in wake-up time” indicates that the wake-up time has changed, and more specifically indicates that the wake-up time has been earlier or later than usual, for example.

Examples of the “situation related to the difference from the reference” include a situation related to body temperature, a situation related to blood pressure, and a situation related to complexion. The situation related to the body temperature indicates that the body temperature was close to or away from the reference value. The situation related to the blood pressure indicates that the blood pressure was close to or away from the reference value. The situation related to the complexion indicates, for example, that the complexion (or an index value indicating the complexion) was close to or away from the usual complexion (the reference value as an index). Each determination may be, for example, a threshold determination for a difference from the reference value. The reference value and the threshold may be set for each user U by a doctor's decision or the like.

Returning to FIG. 3, for example, one or more situations among the situations illustrated in FIG. 4 described above are extracted by the extraction unit 22. Naturally, various situations other than those illustrated in FIG. 4 can also be extracted.

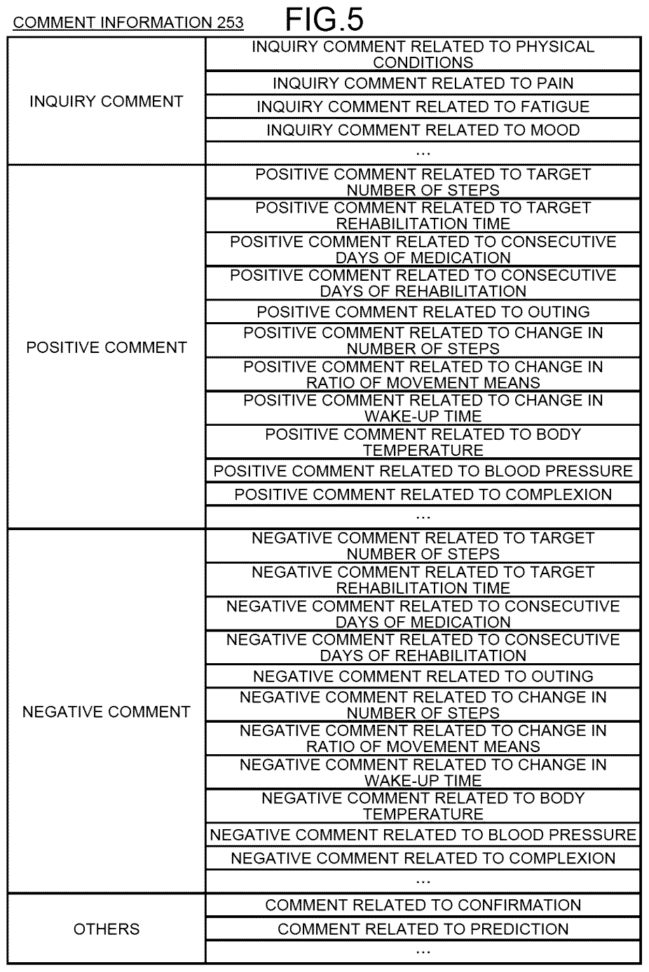

The generation unit 23 generates content including a comment on the situation extracted by the extraction unit 22. The generated content is transmitted to the user terminal 12 and presented to the user U. Details of the presentation will be described below. In the information processing device 2, for example, as illustrated in FIG. 3, information related to various comments is stored in the storage unit 25 as the comment information 253. The comment itself may be stored as the comment information 253, or information in the form of a template of the comment may be stored as the comment information 253. The comment information 253 will be described with reference to FIG. 5.

FIG. 5 is a diagram illustrating an example of the comment information 253. Examples of this include “inquiry comment”, “positive comment”, “negative comment”, and “other comments”.

The “inquiry comment” is a comment for making an inquiry as the name indicates, and includes a comment for inquiring the reason of an extracted situation, for example. Examples of the inquiry comment include an inquiry comment related to physical conditions, an inquiry comment related to pain, an inquiry comment related to fatigue, and an inquiry comment related to mood. Specific examples of these include whether the physical condition is bad, whether there is pain, whether the user is tired, whether the user is sick, and the degree of each symptom. Some examples of modes of the inquiry comment will be described. For example, the inquiry comment may be a dichotomous scale (Yes/No) inquiry comment. A specific example is “Not feeling well?”. The inquiry comment may be a visual analog scale (VAS) type inquiry comment. A specific example is “On a scale of 1 to 10, with 10 being the worst, how would you rate your pain?”. The inquiry comment may be a multiple choice type inquiry comment. A specific example is “Why did you not go out today? Select from: be tired/pain in feet/not feel like going out”.

The comment may use expressions such as a word, onomatopoeia, and the like, which are familiar to the user U. Examples of the inquiry comment in a case where the user U is a child include: “How severe is your pain? Was it sharp pain/throbbing pain/shooting pain/thumping pain/twitching pain?” or the like. The vocabulary and onomatopoeia to be used may be set as appropriate by the healthcare provider C and the member M. The answer by the user U may be performed as voice input.

The positive comment is a favorable comment on the extracted situation, and in this example, includes a favorable comment on each situation illustrated in FIG. 4 described above. Specific examples include a comment indicating that the number of steps or rehabilitation time has reached or is likely to reach the target number of steps or the target rehabilitation time, that daily medication or rehabilitation has been consecutively performed for a predetermined number of days, that an outing has been performed or daily outing has been consecutively performed for a predetermined number of days, that the tendency of an increase in the cumulative number of steps has been heightened, that the ratio of walking movement has increased, that the body temperature or blood pressure has been close to the reference value, that the complexion has been normal, and the like.

The negative comment is an unfavorable comment on the extracted situation, and in this example, includes an unfavorable comment on each situation illustrated in FIG. 4 described above. Specific examples include a comment indicating that the target number of steps or the target rehabilitation time has not been reached or is not likely to be reached, that the number of consecutive days of medication or the number of consecutive days of rehabilitation has not reached a predetermined number of days, that the outing has not been performed or that the outing has not been performed consecutively for a predetermined number of days, that the tendency of the increase in the cumulative number of steps has been lowered, that the ratio of walking movement has decreased, that the body temperature or the blood pressure has been away from the reference value, that the complexion has not been good, and the like.

Examples of other comments include a comment related to confirmation and a comment related to prediction. Specific examples of the comment related to the confirmation include a comment indicating whether the user has taken medicine today, and a comment indicating that the day of hospital visit is coming. Specific examples of the comment related to prediction are comments indicating that it may be sunny tomorrow, and that it would be great to be able to walk more tomorrow.

Content including various comments as described above as examples is generated by the generation unit 23. The comment included in the content is presented to the user U. Here, there are many comments that can be presented for even one situation. On the other hand, the amount of comments that can be presented to the user U is limited. This leads to a prioritization (priority) problem.

Returning to FIG. 3, the calculation unit 24 calculates the priority of the comment. The priority here is the priority of presentation to the user U.

The situation previously extracted by the extraction unit 22 can already be handled as a situation with deviation. As described above with reference to FIG. 4, this is because the reference value, the target value, the threshold, and the like (hereinafter, also referred to as an “extraction determination reference value”) used for the determination reference for extraction are set for each user U. It can be said that the situation extracted by the extraction unit 22 has already been standardized.

Specifically, the deviation value and the priority are referred to as a deviation value D and a priority Pv. For example, when the value indicating the extracted situation is the same as the extraction determination reference value, the deviation value D is 50. As the value deviates from the extraction determination reference value, the deviation value D increases or decreases. Using the situation related to the target number of steps as an example, the extraction determination reference value of the user U who usually walks a lot is set to be high, while the extraction determination reference value of the user U who usually does not walk a lot is set to be low. Therefore, even with the same number of steps (for example, 5000 steps/day), the deviation value D is as low as 40, 35, etc. in the case of the user U who usually walks a lot, whereas the deviation value D is as high as 60, 65, etc. in the case of the user U who usually does not walk a lot. Furthermore, the situation related to the number of consecutive days of rehabilitation will be described as an example. Even in the same number of consecutive days of rehabilitation (for example, 10 consecutive days), the deviation value D is low in the case of the user U who tends to continue rehabilitation for a long time, while the deviation value D is high in the case of the user U who tends to continue rehabilitation for a long time.

For example, the calculation unit 24 calculates the priority Pv of the comment based on the deviation value D as described above. Specifically, the calculation unit 24 calculates the priority Pv of the comment based on a difference between the deviation value D of the situation to be commented and a priority calculation reference value Th. The priority calculation reference value Th is a reference value set for the deviation value D. For example, calculation of the priority Pv uses the following Formula (1). In the formula, i of each variable represents a variable corresponding to the i-th comment. i is an integer of 1 or more.

$${P}_{vi} = {W}_{i} \times \vert {D}_{i} - {T}_{hi} \vert \qquad ( 1 )$$

In the above equation (1), W is a weighting factor. As can be understood, the priority Pv of the comment is calculated such that the more separated the deviation value D from the priority calculation reference value Th, the higher the priority Pv. The comment on the situation in which the deviation value D is away from the priority calculation reference value Th can be preferentially included in the content. Note that parameters related to priority calculation, such as the weighting factor W and the priority calculation reference value Th, can be set as appropriate by the healthcare provider C or the like. For example, by biasing the priority calculation reference value Th, the priority Pv can be made higher when the deviation value D is large than when the deviation value D is small, or conversely, the priority Pv can be made lower when the deviation value D is small than when the deviation value D is large.

More specifically, the calculation unit 24 may perform the calculation such that the more the number of consecutive days of generation (that is, the number of consecutive days of presentation) of the content including the same comment, the lower the priority Pv of the comment. Calculation is performed by using the following Formula (2), for example.

$${P}_{vi} = {W}_{i} \times \vert {D}_{i} - {T}_{hi} \vert - n \times {W}_{i} \qquad ( 2 )$$

In the above Formula (2), n is the number of consecutive days of generation of the content including the comment. As compared with Formula (1) described above, the priority Pv is lowered by a value obtained by multiplying the number of consecutive days of generation n by the individual weighting factor W. This makes it possible to suppress generation and presentation of content including similar comments for consecutive days. Note that the two weighting factors W in Formula (2) may be set to different values.

More specifically, in a case where it is necessary to delve into the extracted situation based on the answer given by the user U to the inquiry comment, the calculation unit 24 may calculate the priority so as to increase the priority Pv of another inquiry comment regarding the situation. Otherwise, specifically, in a case where there is no need to perform delving, the priority may be calculated such that the priority Pv of the another inquiry comment becomes low. Calculation is performed by using the following Formula (3), for example.

$${P}_{vi} = {W}_{i} \times \vert {D}_{i} - {T}_{hi} \vert - n \times {W}_{i} + {\sum }_{j} {α}_{ij} - {\sum }_{j} {β}_{ij} \qquad ( 3 )$$

In the above Formula (3), α and β are scores (correction values and the like), and can be set as appropriate by the healthcare provider C similarly to the weighting factor W and the priority calculation reference value Th. j represents the number (such as ID of the item) attached to the related comment of the same day. That is, i in the score α and the score β indicates the number attached to the corresponding comment, and j indicates the number attached to the comment on the affecting side. For example, the score α₅₂ means that the score α of the fifth comment is affected by the answer to the second inquiry comment. As compared with Formula (2) described above, the priority Pv increases or decreases by the score α or the score β. The priority Pv is calculated using Formula (3) set to increase the score α in a case where it is necessary to delve into the situation. When it is not necessary to delve into the situation, the priority Pv is calculated using Formula (3) set to increase the score β. A comment about a situation that need delving can be preferentially included in the content.

The necessity of delving may be determined based on a rule. For example, in a case where an inquiry comment for inquiring the reason why the number of steps was small is presented to the user U and an answer indicating that the user U gets tires easily is obtained from the user U, it is determined that delving is necessary to grasp a more detailed situation. The priority is calculated so as to increase the priority Pv of the inquiry comment for delving, for example, “whether or not there is chest pain?”. Conversely, in a case where an inquiry comment for inquiring the reason why the number of times of outing was small is presented by the user U and an answer indicating that it was necessary to look after the house is obtained from the user U, it is determined that there is no need to perform delving for grasping a more detailed situation. Priority calculation is performed to decrease the priority Pv of the inquiry comment for delving.

Incidentally, the calculation unit 24 may perform priority calculation such that the priority Pv of the positive comment becomes higher as the ratio of the negative comment to the comment included in the content generated by the generation unit 23 so far becomes larger than the ratio of the positive comment. In the example of the above Formula (3), the priority Pv of the positive comment is calculated using the Formula (3) set to increase the score α. This makes it possible to suppress the ratio of negative comments from becoming too small.

Returning to FIG. 3, the generation unit 23 generates content including a comment having the high priority Pv calculated by the calculation unit 24. For example, the unit generates content including one or more comments selected in descending order of the priority Pv and content including one or more comments with the priority Pv of a predetermined value (threshold) or more. The number to be selected or the number to be set as a predetermined value may be appropriately set according to a presentation time of the content to the user U, for example.

In addition to the comment, the content may also include information related to a situation to be commented, for example, information such as sensing data corresponding to the situation. The content is transmitted to the user terminal 12 by the communication unit 21.

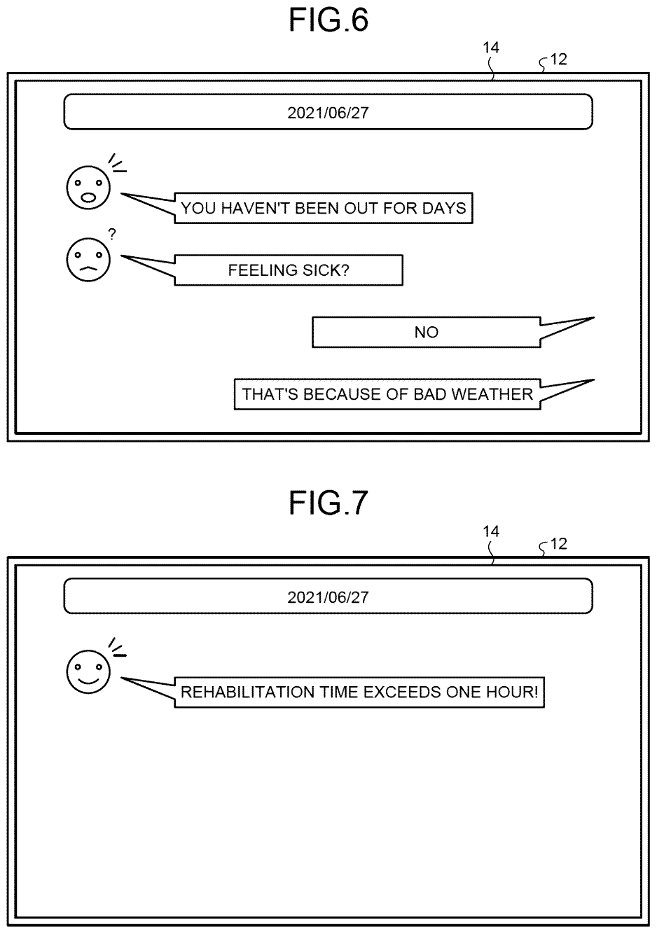

The communication unit 13 of the user terminal 12 receives content from the information processing device 2. The user interface unit 14 presents content. Furthermore, the user interface unit 14 receives an answer given by the user U to the inquiry comment included in the content. This will be described with reference to FIGS. 6 and 7.

FIGS. 6 and 7 are diagrams illustrating examples of content presentation. In this example, a message included in the content is presented in a chat form. These are merely examples, and content including various comments may be presented to the user U in various modes.

The example illustrated in FIG. 6 presents a negative comment related to outing “You haven't been out for days” and an inquiry comment related to physical condition “Feeling sick?” are presented. In response to this, the answer “No” and “That's because of bad weather” are obtained from the user U.

In the example illustrated in FIG. 7, a positive comment related to the target rehabilitation time “Rehabilitation time exceeds one hour!” is displayed. In this case, it would be no problem to have no particular answer from the user U. Praising the user U will have an effect of encouraging the user U to continue rehabilitation.

Returning to FIG. 3, the answer from the user U to the inquiry comment is transmitted from the communication unit 13 to the information processing device 2. The communication unit 21 of the information processing device 2 receives the answer. According to the answer, the priority Pv of the comment is calculated again (recalculated) by the calculation unit 24, and the content including the comment having the high priority Pv is generated by the generation unit 23 and presented to the user U. The series of processing is repeated, for example, until a certain period elapses from the start of presentation of the content or the priority Pv of the comment not presented yet becomes a threshold or less.

When the presentation of the content is finished, the communication unit 21 of the information processing device 2 transmits result information obtained by the processing in the information processing device 2 to the healthcare provider terminal 3 and the community member terminal 4. The result information includes content presented to the user U, an answer given by the user U to an inquiry comment included in the content, and the like. Naturally, the result information may be stored and saved in the storage unit 25 of the information processing device 2.

The healthcare provider terminal 3 includes a communication unit 31, a user interface unit 32, and a storage unit 33. Examples of the information stored in the storage unit 33 include medical information 331. The medical information 331 includes, for example, medical record information regarding the user U and is used for purposes such as diagnosis of the user U by the healthcare provider C.

The communication unit 31 communicates with other devices and the like. For example, the communication unit 31 receives result information from the information processing device 2. Furthermore, the communication unit 31 transmits parameters to the user terminal 12.

The user interface unit 32 receives an operation of the healthcare provider terminal 3 by the healthcare provider C and presents (displays or the like) information to the healthcare provider C. For example, the user interface unit 32 receives an input of a parameter. Examples of the parameter include values such as the extraction determination reference value used for extraction by the extraction unit 22 of the information processing device 2 and the priority calculation reference value Th used for calculation by the calculation unit 24, described above. Such a parameter is set in the healthcare provider terminal 3 and transmitted to the information processing device 2. In the information processing device 2, information including a parameter is stored in the storage unit 25 as the parameter information 252. Furthermore, the user interface unit 32 presents the result information. Based on the presented result information, the healthcare provider C can grasp not only the user U's situation grasped from the sensing data, but also the detailed situation of the user U grasped from the content presented to the user U and the answer obtained from the user U. For example, in the example of FIG. 6 described above, it is possible to grasp that the reason of consecutive days without outing is not their poor physical condition but bad weather. As necessary, the medical information 331 is updated in the form of addition of medical record information, etc.

The community member terminal 4 includes a communication unit 41 and a user interface unit 42. The communication unit 41 communicates with other devices and the like. For example, the communication unit 41 receives result information from the information processing device 2.

The user interface unit 42 receives an operation of the community member terminal 4 by the member M and presents information to the member M. For example, the user interface unit 42 presents the result information. The result information is shared with the member M. The community member terminal 4 may be able to perform parameter setting, similarly to the healthcare provider terminal 3.

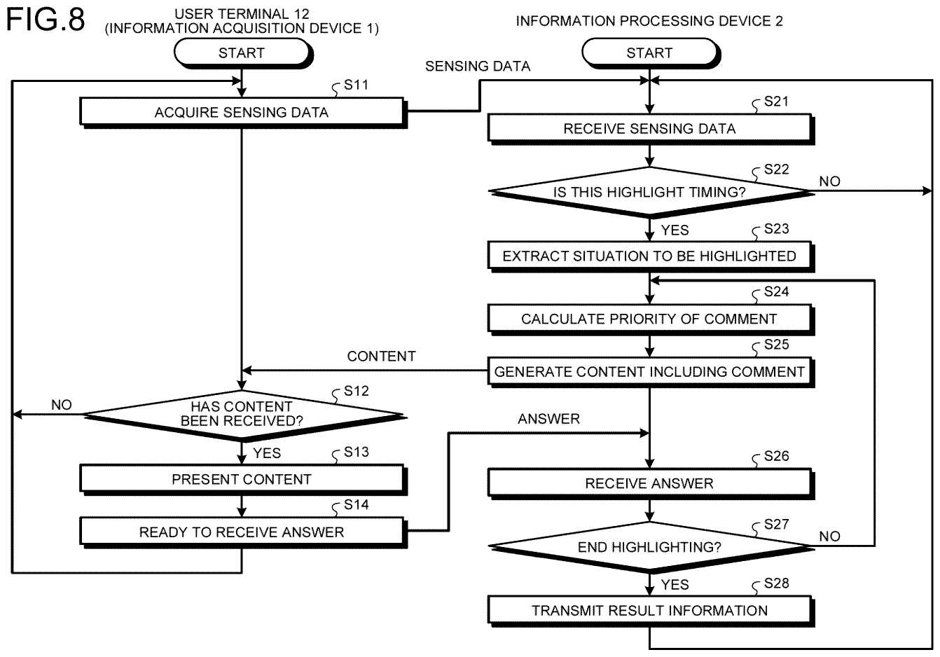

FIG. 8 is a flowchart illustrating an example of processing (information processing method) executed in the information processing system. Description overlapping with the above description will be omitted as appropriate. The user terminal 12 may be appropriately replaced with the information acquisition device 1 within a range without contradiction.

In Steps S11 and S12, the user terminal 12 continues to acquire sensing data until the content is received from the information processing device 2. The communication unit 13 of the user terminal 12 receives the sensing data from the sensor 11 and transmits the sensing data to the information processing device 2. In a case where the user terminal 12 has received the content (Step S12: Yes), the processing proceeds to Step S13, and otherwise (Step S12: No), the processing of Step S11 is repeated.

In Steps S21 and S22, the information processing device 2 continues to receive sensing data until the highlight timing comes. The communication unit 21 of the information processing device 2 receives the sensing data from the information acquisition device 1. The extraction unit 22 determines whether it is a highlight timing. For example, when a predetermined time of the day comes, it is determined that a highlight timing has come. In a case where the highlight timing has come (Step S22: Yes), the information processing device 2 proceeds to the processing of Step S23. Otherwise, the information processing device 2 repeats the processing of Step S21.

In Step S23, the information processing device 2 extracts a situation to be highlighted. For example, the extraction unit 22 of the information processing device 2 extracts the situation of to be highlighted from among the user U's situations grasped from the sensing data received so far after the lapse of the highlight timing of the previous day.

In Step S24, the information processing device 2 calculates the priority of the comment. The calculation unit 24 of the information processing device 2 calculates the priority Pv of the comment regarding the situation extracted by the extraction unit 22.

In Step S25, the information processing device 2 generates content including a comment. The generation unit 23 of the information processing device 2 generates content including a comment having the high priority Pv calculated by the calculation unit 24. The generated content is transmitted to the user terminal 12. In response to reception of the content (Step S12: Yes), the user terminal 12 proceeds to the processing of Step S13.

In Step S13, the user terminal 12 presents content. The user interface unit 14 of the user terminal 12 presents content including a comment. For example, as illustrated in FIG. 6 described above, an inquiry comment or the like is presented.

In Step S14, the user terminal 12 is ready to receive an answer. For example, the answer given by the user U as described above with reference to FIG. 6 is received and transmitted to the information processing device 2. Thereafter, the user terminal 12 returns the processing to Step S11.

In Step S26, the information processing device 2 receives the answer. The communication unit 21 of the information processing device 2 receives the answer from the user terminal 12.

In Step S27, the information processing device 2 determines whether to end the highlighting. For example, in a case where a predetermined condition is satisfied, the extraction unit 22 of the information processing device 2 determines to end the highlighting. The predetermined condition is satisfied in a case where a certain period elapses from the start of presentation of the content or the priority Pv of the comment not presented yet becomes a threshold or less. In the case of ending the highlighting (Step S27: Yes), the information processing device 2 proceeds to the processing of Step S28. Otherwise (Step S27: No), the information processing device 2 returns to the processing of Step S24. Hereinafter, Step S24 will be described first, and then Step S28 will be described.

When the processing returns to Step S24, the processing of Step S24, Step S25, Step S12 to Step S14, and Step S26 is executed again. The priority Pv of the comment is recalculated. For example, by using Formula (3) described above, priority calculation is performed such that the priority Pv of the presented comment becomes low. Content including another comment having a high priority Pv is generated and presented to the user U.

On the other hand, when the processing proceeds to Step S28, the information processing device 2 transmits result information. The communication unit 21 of the information processing device 2 transmits result information including the content and the answer given by the user U so far to the healthcare provider terminal 3 and the community member terminal 4. The result information is presented to the healthcare provider C in the healthcare provider terminal 3 and to the member M in the community member terminal 4. This makes it possible to grasp the daily situation of the user U that is difficult to grasp only from the sensing data.

After the processing of Step S28 is completed, the information processing device 2 returns to the processing of Step S21.

### 2. Modification

The disclosed technology is not limited to the above embodiments. Some modifications will be described.

The above embodiment is an exemplary case where content is presented in a chat format. However, the content may be presented in various forms other than a chat form. For example, only the comment may be presented simply, or conversely, the content may be presented in a more expanded mode. An example of the latter will be described.

For example, the content may be presented in a mode imitating a television program, coverage, or the like (content presentation by a metaphor). The content may include an avatar that makes a conversation including an utterance of a comment or makes a reaction. The avatar may be construed to include a virtual person such as a character. The use of the avatar increases the possibility of assisting or encouraging the user U to give an answer. The length of the program and the timing of the reaction of the avatar (waiting time or the like) may be flexibly set. There may be provided a skip function. Various metaphors, such as news programs, may be used in program formats. This increases the possibility of assisting or encouraging the user U to give an answer.

The avatar may be appropriately selected, modified, or the like by the user U or the healthcare provider C. Characters popular with children may be made available. A character with a limited period such as a stamp may be made available, or there may be a billing element. The utterance of the avatar or its ending may be expressed so as to reflect the personality of the avatar.

The avatar interacting with the user U may be a plurality of avatars. This makes it possible to reduce pressure felt in a case of a medical interview performed one on one. By assigning a role to each avatar, it is possible to achieve both a role of a host of the program, a role to add tastes, and the like. It is also possible to impart various dramatic effects to the entire content by a dialog between avatars, assignment for each scene (corner or the like), or the like. In a case where there is one avatar interacting with the user U, each scene may be adjusted so that it can be understood that one avatar has a plurality of roles.

There may be provided a real-time response function. For example, the avatar may perform cheering for the user U at the time of rehabilitation or the like. Such a message or the like related to the cheering is also an example of the content. A summary of the content and a state (image or the like) at the time of the answer given by the user U may be presented to the healthcare provider C and the member M via the healthcare provider terminal 3 and the community member terminal 4.

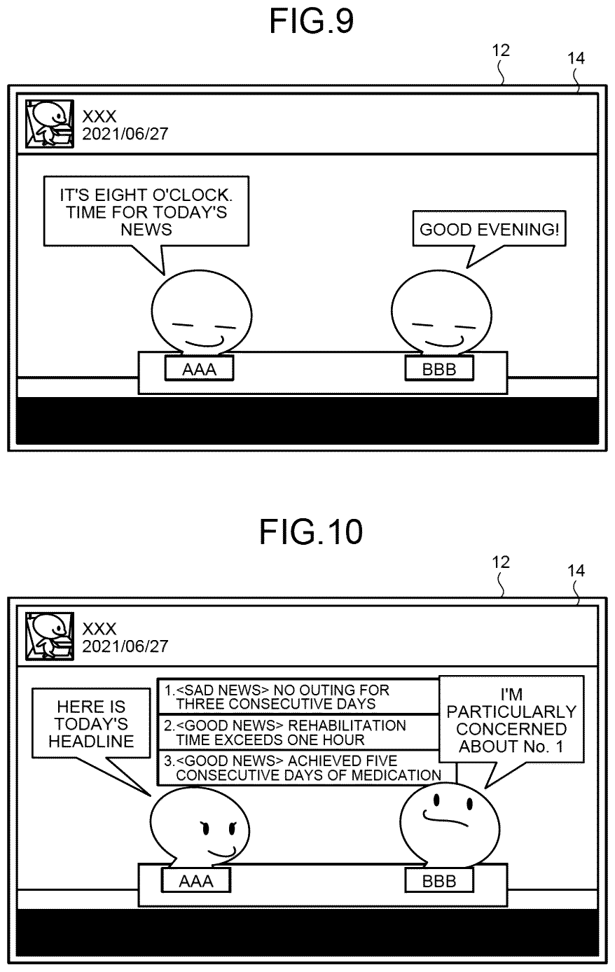

As an example, content presentation in a mode simulating a television program will be described with reference to FIGS. 9 to 15.

FIGS. 9 to 15 are diagrams illustrating examples of content presentation. In this example, content is presented like a news program. A plurality of avatars each imitating a newscaster (for example, corresponding to an avatar of the healthcare provider C) chairs the program while performing conversation and reaction. The name of the user U is schematically illustrated as “xxx” together with an icon of the user U in the upper left of the screen. The date when the content was presented (inquiry date) is also displayed, leading to acquisition of information regarding the date when the inquiry comment was presented. The avatar encourages the user U to answer with a natural context according to the conversation. Furthermore, a state in which the user U answers is displayed as an image or a video like an interview with a star athlete. At that time, the voice of the user U may be collected, and the content of the utterance of the user U may be estimated based on the voice recognition result or the like.

Specifically, an opening speech as illustrated in FIG. 9 is performed, and thereafter comments 1 to 3 according to the highlighted situation are presented as a list as illustrated in FIG. 10. The first comment “1. <Sad news> No outing for three consecutive days” is a negative comment. The second comment “2. <Good news> Rehabilitation time exceeds one hour” and the third comment “3. <Good news> Achieved five consecutive days of medication” are positive comments. The program is directed so as to delve into the situation of the target of the negative comment.

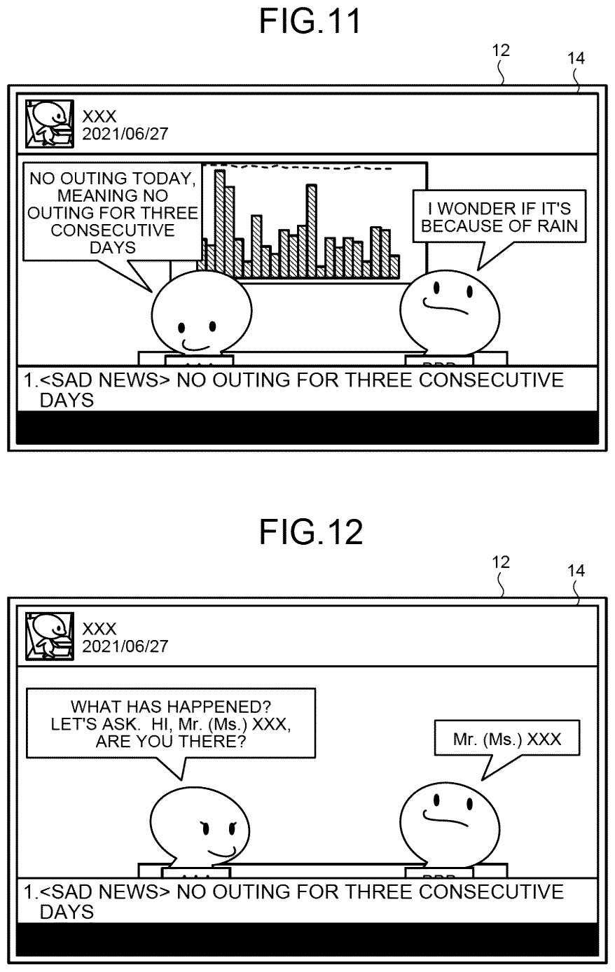

As illustrated in FIG. 11, in addition to the similar negative comment “No outing today, meaning no outing for three consecutive days”, an inquiry comment “I wonder if it's because of rain” is displayed. In addition, related data (bar graph in this example) is also displayed. Subsequently, as illustrated in FIG. 12, a call to get attention of the user U is made, such as “What has happened? Let's ask. Hi, Mr. (Ms.) xxx! Are you there?” etc.

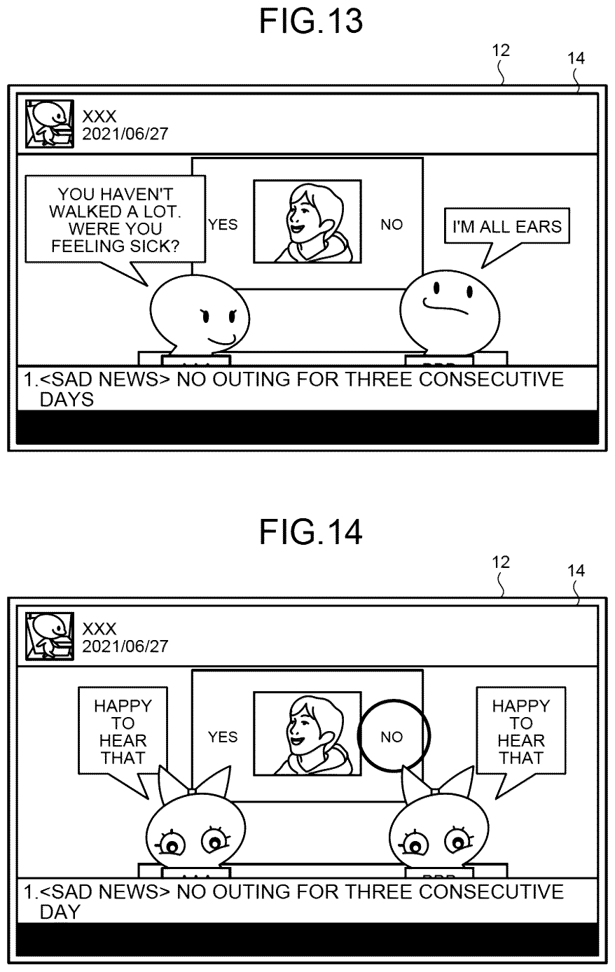

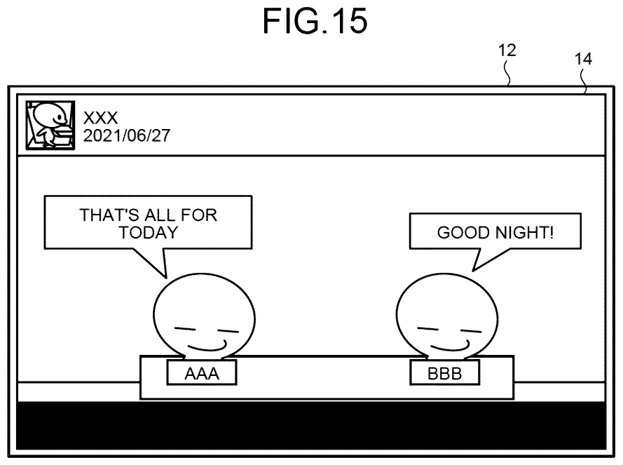

As illustrated in FIG. 13, an inquiry comment “You haven't walked a lot. Were you feeling sick?” is presented. As illustrated in FIG. 14, the answer “No” of the user U is displayed together with an image (video) including the state of the user U who is making the answer. The display also includes occurrence of reactions of avatars, such as “I am all ears.” and “Happy to hear that”. The program is directed with a change in the inquiry comment and the like according to the answer given by the user U. When a certain period of time has elapsed or when all answers to the inquiry comment with high priority Pv are obtained, a final greeting as illustrated in FIG. 15 is made to end the program. Note that an image such as a screen at the time of content presentation may also be included in or saved in the result information. In particular, including the date information in the image increases the saving value of the image. For example, a screen image as illustrated in FIG. 10 may be saved as a representative image of the day.

In addition to the modes illustrated in FIGS. 9 to 15 described above, content presentation and the like may be performed in various modes. For example, it is allowable to randomly generate an utterance by avatars or the like so as to provide an accent. Examples of utterances to provide an accent include interjections as affirmation, speech using a metaphor. Current news items such as weather and news may be obtained by crawling. It is also possible to use not only speech but also funny faces, strange sounds and the like. The avatar may make an utterance including a prediction element. Examples of the prediction element include weather prediction indicating that it may be sunny tomorrow and situation prediction indicating that it would be great to be able to walk more tomorrow. The success or failure of the prediction does not particularly matter.

The sensing data related to the user U at the time of the answer may also be included in the result information and presented to the healthcare provider C or the like. For example, quantitative information of the sensing data of the vital sensor may be included in the result information. Such information is also information useful for diagnosis or the like by the healthcare provider C. Information such as the tone of the voice of the user U at the time of answer may also be included.

Information such as the heartbeat and respiration of the user U may be grasped from the sensing data of the image sensor. For example, the image of the face of the user U makes it possible to grasp information such as a face-image-based heart rate and the respiratory rate. As a specific technique, for example, the technique described in Patent Literature 3 may be used.

As described above, examples of the user terminal 12 include a smartphone and a television. In the case of a mobile communication terminal such as a smartphone, it is easy to distribute an application and the like. In the case of a fixed terminal device such as a television, it is also possible to perform control such that the screen display is automatically turned on at the timing of starting the presentation of information to be highlighted, leading to an increased possibility of improving the user experience such as increased real-time sensation and an immersed feeling.

At least some functions of the information processing device 2 may be provided in the user terminal 12. For example, some or all of the functions of the communication unit 21, the extraction unit 22, the generation unit 23, the calculation unit 24, and the storage unit 25 of the information processing device 2 may be installed on the user terminal 12. In a case where all of the functions of the information processing device 2 are mounted on the user terminal 12, the user terminal 12 also functions as the information processing device 2.

The sensing data of the sensor 11 may be directly transmitted to the information processing device 2 without passing through the user terminal 12. The configuration is not limited thereto, and each function of the information processing system 100 may be provided in a distributed manner in various devices, terminals, and the like within a range in which the functions of the information processing system 100 can be implemented.

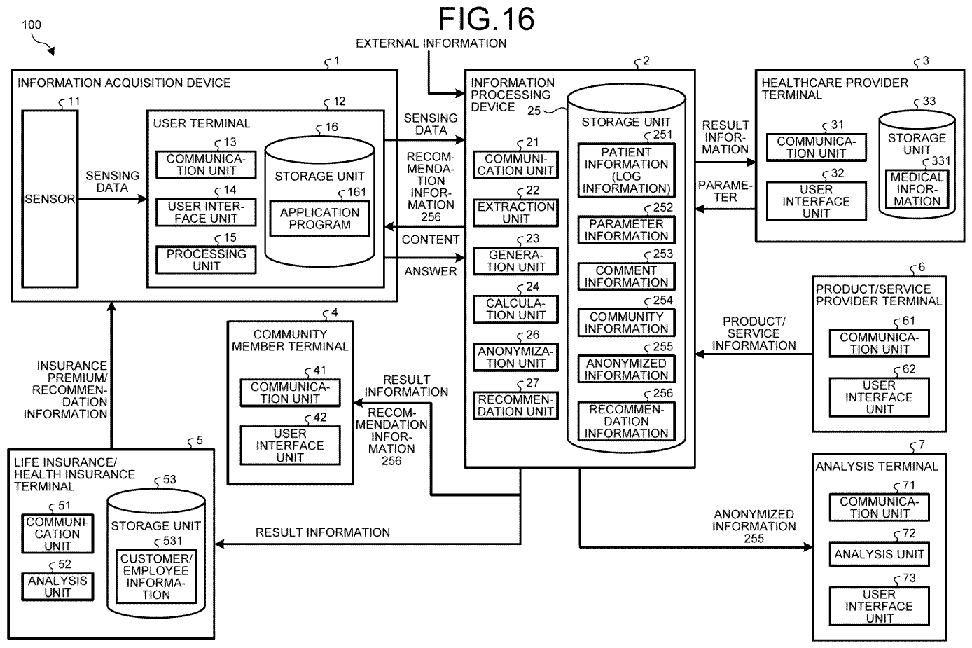

It is also possible to further utilize the information processing system 100 including result information obtained by processing in the information processing device 2. This will be described with reference to FIG. 16.

FIG. 16 is a diagram illustrating an example of further utilization of result information and the information processing system. In addition to the information acquisition device 1, the information processing device 2, the healthcare provider terminal 3, and the community member terminal 4 described above, there are provided, as examples, a life insurance/health insurance terminal 5, a product/service provider terminal 6, and an analysis terminal 7.

The life insurance/health insurance terminal 5 is a terminal used by an insurance company, a health insurance company, or the like. The communication unit 21 of the information processing device 2 transmits the result information to the life insurance/health insurance terminal 5.

The life insurance/health insurance terminal 5 includes a communication unit 51, an analysis unit 52, and a storage unit 53. Examples of information stored in the storage unit 53 include customer/employee information 531. The customer/employee information 531 includes information related to life insurance, health insurance, or the like, regarding the user U.

The communication unit 51 receives result information from the information processing device 2. The analysis unit 52 analyzes the result information (for example, the situation of the user U) and specifies (calculates) an insurance premium and a reward. Specifying operation may be performed via work, determination, and the like of an employee or the like of a life insurance company or a health insurance company. It is also allowable to perform reduction in insurance premium, a change to a limited plan, and the like. The communication unit 51 transmits the specified insurance premium/reward information to the user terminal 12. The insurance premium/reward information is presented by the user interface unit 14 of the user terminal 12.

The result information may be anonymized and used for product or service recommendation or data analysis. In the example illustrated in FIG. 16, the information processing device 2 further includes an anonymization unit 26 and a recommendation unit 27. The storage unit 25 also stores anonymized information 255 and recommendation information 256.

The anonymization unit 26 anonymizes the result information to generate anonymized information 255. The anonymized information 255 describes anonymized personal information and result information in association with each other.

The product/service provider terminal 6 is a terminal used by a company or the like that provides a product/service. Examples of the product include a wheelchair, a walking aid, a rehabilitation device, a health food product, and health equipment. An example of the service is a health application or the like executable on the user terminal 12.

The product/service provider terminal 6 includes a communication unit 61 and a user interface unit 62. For example, product/service information for associating anonymized result information with a product/service is input or generated via the user interface unit 62. The communication unit 61 transmits the product/service information to the information processing device 2.

The communication unit 21 of the information processing device 2 receives product/service information from the product/service provider terminal 6. Based on the product/service information from the product/service provider terminal 6, the recommendation unit 27 of the information processing device 2 generates recommendation information 256 including information regarding a product or service to be recommended to the user U. The communication unit 21 transmits the recommendation information 256 to the user terminal 12 and the community member terminal 4. The recommendation information 256 is presented by the user interface unit 14 of the user terminal 12 and the user interface unit 42 of the community member terminal 4.

In addition, the communication unit 21 of the information processing device 2 transmits anonymized information 255 to the analysis terminal 7. The analysis terminal 7 is, for example, a terminal used by a company that provides the above-described product/service, a pharmaceutical company that performs clinical development, or the like. The analysis terminal 7 includes a communication unit 71, an analysis unit 72, and a user interface unit 73.

The communication unit 71 receives the anonymized information 255 from the information processing device 2. The analysis unit 72 performs data analysis based on the anonymized information 255. The analysis may involve work, judgment, or the like of an employee or the like of the company. The user interface unit 73 presents information related to data analysis and the like. Examples of the analysis include analysis of a user group of products such as health food products and health equipment, data analysis for clinical development, and the like.

The information processing device 2, the life insurance/health insurance terminal 5, the product/service provider terminal 6, and the analysis terminal 7 illustrated in FIG. 16 may also be components of the information processing system 100.

### 3. Hardware Configuration Example

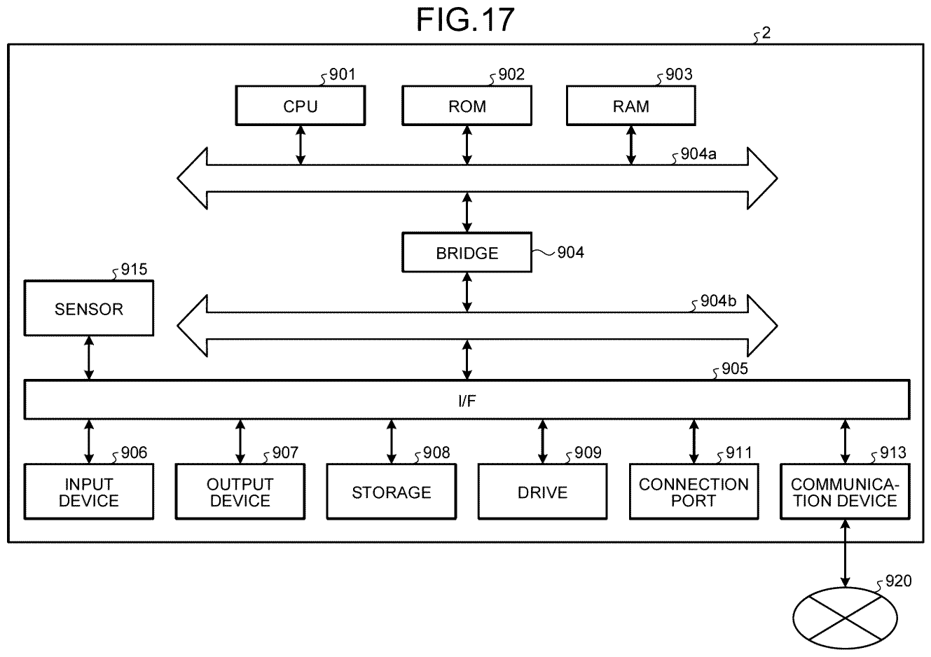

FIG. 17 is a block diagram illustrating an example of a hardware configuration. Hereinafter, the information processing device 2 will be described as an example. Similar description is possible regarding the user terminal 12, the healthcare provider terminal 3, the community member terminal 4, and the like. Various types of processing performed by the information processing device 2 are implemented by cooperative operations of software and hardware described below.

As illustrated in FIG. 17, the information processing device 2 includes a central processing unit (CPU) 901, read only memory (ROM) 902, random access memory (RAM) 903, and a host bus 904a. The information processing device 2 further includes a bridge 904, an external bus 904b, an interface 905, an input device 906, an output device 907, a storage device 908, a drive 909, a connection port 911, a communication device 913, and a sensor 915. The information processing device 2 may include a processing circuit such as a DSP or an ASIC instead of or in addition to the CPU 901.

The CPU 901 functions as an arithmetic processing device and a control device, and controls the overall operation in the information processing device 2 according to various programs. In addition, the CPU 901 may be a microprocessor. The ROM 902 stores programs and calculation parameters used by the CPU 901. The RAM 903 temporarily stores a program used in the execution of the CPU 901, parameters that change appropriately in the execution, or the like. The CPU 901 can embody units of the information processing device 2, such as the extraction unit 22, the generation unit 23, the calculation unit 24, for example.

The CPU 901, ROM 902, and RAM 903 are connected to each other by the host bus 904a including a CPU bus or the like. The host bus 904a is connected to the external bus 904b such as a Peripheral Component Interconnect/Interface (PCI) bus via the bridge 904. Note that the host bus 904a, the bridge 904, and the external bus 904b need not have a configuration separated from each other, and may be implemented in a single configuration (for example, in one bus).

The input device 906 is actualized by a device to which the technician inputs information, such as a mouse, a keyboard, a touch panel, a button, a microphone, a switch, and a lever. Furthermore, the input device 906 may be, for example, a remote control device using infrared rays or other radio waves, or an externally connected device such as a mobile phone or a PDA that supports the operation of the information processing device 2. Furthermore, the input device 906 may include, for example, an input control circuit that generates an input signal based on the information input by the technician using the above input means and outputs the input signal to the CPU 901. By operating the input device 906, the technician can input various data to the information processing device 2 and give an instruction on the processing operation.

The output device 907 is formed by a device capable of visually or audibly notifying the technician of acquired information. Examples of such devices include display devices such as CRT display devices, liquid crystal display devices, plasma display devices, EL display devices, and lamps, audio output devices such as speakers and headphones, and printer devices.

The storage device 908 is a device for storing data. The storage device 908 is implemented by, for example, a magnetic storage device such as an HDD, a semiconductor storage device, an optical storage device, an optical magnetic storage device, or the like. The storage device 908 may include a recording medium, a recording device that records data on the storage medium, a reading device that reads data from the storage medium, a deleting device that deletes the data recorded on the storage medium, and the like. This storage device 908 stores programs executed by the CPU 901, various data, as well as various data acquired from the outside, and the like. The storage device 908 can embody, for example, the storage unit 25 or the like of the information processing device 2.

The drive 909 is a reader/writer for a storage medium, and is built in or externally connected to the information processing device 2. The drive 909 reads information recorded on a removable storage medium such as a mounted magnetic disk, optical disk, magneto-optical disk, or semiconductor memory, and outputs the read information to the RAM 903. The drive 909 can also write information to the removable storage medium.

The connection port 911 is an interface connected to an external device, and is a connecting port to an external device capable of transmitting data by, for example, a universal serial bus (USB).

The communication device 913 is, for example, a communication interface formed by a communication device or the like for connecting to a network 920 (for example, corresponding to the network N in FIG. 1). The communication device 913 is, for example, a communication card for wired or wireless Local Area Network (LAN), Long Term Evolution (LTE), Bluetooth (registered trademark), Wireless USB (WUSB), or the like. Furthermore, the communication device 913 may be a router for optical communication, an Asymmetric Digital Subscriber Line (ADSL) router, a modem for various communications, or the like. The communication device 913 can transmit and receive signals or the like to and from the Internet and other communication devices in accordance with a predetermined protocol such as TCP/IP. The communication device 913 can embody, for example, the communication unit 21 or the like of the information processing device 2.

The sensor 915 may include at least some of the sensors included in the sensor 11.

The network 920 is a wired or wireless transmission path for information transmitted from a device connected to the network 920. For example, the network 920 may include a public network such as the Internet, a telephone network, and a satellite communication network, or various local area networks (LANs) including Ethernet (registered trademark), wide area networks (WANs), or the like. Furthermore, the network 920 may include a dedicated network such as an Internet protocol-virtual private network (IP-VPN).

The hardware configuration example capable of implementing the functions of the information processing device 2 has been described above. Each of the above-described components may be implemented by using a general-purpose member, or may be implemented by hardware devices specialized for the function of individual components. Accordingly, it is possible to appropriately change the hardware configuration to be used according to the technical level at the time of conducting the present disclosure.

Incidentally, it is possible to create a computer program for implementation of individual functions of the information processing device 2 as above and possible to install the created program on a PC or the like. Furthermore, it is also possible to provide a computer-readable recording medium storing such a computer program. Examples of the recording medium include a magnetic disk, an optical disk, a magneto-optical disk, a flash drive, or the like. Furthermore, the computer program described above may be distributed via a network, for example, without using a recording medium.

It should be noted that the embodiments and modifications disclosed herein are merely illustrative in all respects and are not to be construed as limiting. The above-described embodiments and modifications can be omitted, replaced, and changed in various forms without departing from the scope and spirit of the appended claims. For example, the above-described embodiments and modifications may be combined in whole or in part, and embodiments other than the above-described embodiments and modifications may be combined with the above-described embodiments or modifications. Furthermore, the effects of the present disclosure described in the present specification are merely illustrative, and other effects may be provided.

A technical category embodying the above technical idea is not limited. For example, the above-described technical idea may be embodied by a computer program for causing a computer to execute one or a plurality of procedures (steps) included in a method of manufacturing or using the above-described device. In addition, the above-described technical idea may be embodied by a computer-readable non-transitory recording medium in which such a computer program is recorded.

### 4. Exemplary Effects

The technology described above is specified as follows, for example. One of the disclosed technologies is an information processing method. As described with reference to FIGS. 1 to 8 and the like, the information processing method uses a procedure in which the information acquisition device 1 receives the sensing data of the sensor 11 provided for the user U being a patient (Step S21), extracts a situation to be highlighted from user U's daily situations grasped from the received sensing data (Step S23), and generates content including a comment on the extracted situation (Step S25). The comment includes an inquiry comment.

According to the above information processing method, the situation to be highlighted is extracted from the user U's daily situations grasped from the sensing data, and the content including the inquiry comment regarding the situation is generated. Based on the answer given by the user U to the inquiry comment, it is possible to grasp the daily situation of the user U (patient), which is difficult to grasp only from the sensing data.

As described with reference to FIG. 5 and the like, the comment may include an inquiry comment for inquiring the reason of the extracted situation. This makes it possible to grasp the reason for the situation to be highlighted.

As described with reference to FIG. 5 and the like, the inquiry comment may include at least one of a comment related to a physical condition, a comment related to pain, a comment related to fatigue, and a comment related to mood. For example, the daily detailed situation of the user U can be grasped based on such an inquiry comment.

As described with reference to FIG. 4 and the like, the situation to be highlighted may include at least one of a situation related to occurrence of a specific behavior, a situation related to a behavioral change, and a situation related to a difference from a reference. The specific behavior may include at least one of an achievement target behavior and a consecutive daily behavior. The achievement target behavior may include at least one of the target number of steps and the target rehabilitation time. The consecutive daily behavior may include at least one of the number of consecutive days of medication and the number of consecutive days of rehabilitation. The behavioral change may include at least one of a change in the number of steps, a change in a ratio of movement means, and a change in wake-up time. The situation related to the difference from the reference may include at least one of a situation related to body temperature, a situation related to blood pressure, and a situation related to complexion. For example, by extracting such a situation as a situation to be highlighted, it is possible to grasp the daily detailed situation of the user U.

As described with reference to FIGS. 3 and 8 and Formulas (1) to (3) and the like, in the information processing method, the information processing device 2 may calculate the priority Pv of the comment (Step S24) and generate the content including the comment having the high calculated priority Pv (Step S25). The comment to be presented to the user U can be preferentially included in the content in this manner, for example.

As described with reference to the Formulas (1) to (3) and the like, the priority Pv of the comment may be calculated based on the difference between the deviation value D of the situation to be commented and the priority calculation reference value Th. The comment on the situation in which the deviation value D is away from the priority calculation reference value Th can be preferentially included in the content.

As described with reference to the Formulas (2) and (3) and the like, the priority Pv of the comment may be calculated such that the more the number of consecutive days of generation of the content including the same comment, the lower the priority Pv. It is possible to suppress generation of content including similar comments for consecutive days.

As described with reference to Formula (3) and the like, in a case where it is necessary to delve into the extracted situation based on the answer given by the user U to the inquiry comment, the priority Pv of the comment may be calculated such that the priority Pv of another inquiry comment regarding the situation becomes high, and otherwise, specifically in a case where there is no need to perform delving, the priority Pv of the comment may be calculated such that the priority Pv of the another inquiry comment becomes low. A comment about a situation that need delving can be preferentially included in the content.

As described with reference to FIG. 5, Formula (3), and the like, the comment includes a positive comment and a negative comment, regarding the extracted situation, and the priority Pv of the comment may be calculated such that the greater the ratio of the negative comment to the comment included in the content generated so far than the ratio of the positive comment, the higher the priority Pv of the positive comment. This makes it possible to suppress the ratio of negative comments from becoming too small.

As described with reference to FIGS. 9 to 15 and the like, the content may include an avatar that utters a comment. The use of the avatar increases the possibility of assisting or encouraging the user U to give an answer.

The information processing system 100 described with reference to FIGS. 1 to 7 and the like is also one of the disclosed technologies. The information processing system 100 includes: the information acquisition device 1 that acquires sensing data of the sensor 11 provided for the user U being a patient; and the information processing device 2 that extracts a situation to be highlighted from the user U's daily situations grasped from the sensing data acquired by the information acquisition device 1 and generates content including a comment (including an inquiry comment) on the extracted situation. With such an information processing system 100, it is also possible to grasp the daily situation of the user U (patient), which is difficult to grasp only from the sensing data. The information processing system 100 may further include the user terminal 12 that presents content generated by the information processing device 2 to the user U.

The information processing device 2 described with reference to FIGS. 1 to 7 and the like is also one of the disclosed technologies. The information processing device 2 includes the extraction unit 22 that extracts a situation to be highlighted from among daily situations of the user U being a patient, the situations grasped from sensing data of a sensor 11 provided for the user U; and the generation unit 23 that generates content including a comment (including an inquiry comment) on the situation extracted by the extraction unit 22. With such an information processing device 2, it is also possible to grasp the daily situation of the user U (patient), which is difficult to grasp only from the sensing data.

Note that the present technology can also have the following configurations.
(1) An information processing method to be implemented by an information processing device, the method comprising:
   receiving sensing data obtained by a sensor provided for a user being a patient;
   extracting a situation to be highlighted among daily situations of the user, the situations grasped from the received sensing data; and
   generating content including a comment on the extracted situation,
   wherein the comment includes an inquiry comment.
(2) The information processing method according to (1),
   wherein the comment includes an inquiry comment for inquiring a reason of the extracted situation.
(3) The information processing method according to (1) or (2),
   wherein the inquiry comment includes at least one of a comment related to a physical condition, a comment related to pain, a comment related to fatigue, and a comment related to mood.
(4) The information processing method according to any one of (1) to (3),
   wherein the situation to be highlighted includes at least one of a situation related to occurrence of a specific behavior, a situation related to a behavioral change, and a situation related to a difference from a reference.
(5) The information processing method according to (4),
   wherein the specific behavior includes at least one of an achievement target behavior and a consecutive daily behavior.
(6) The information processing method according to (5),
   wherein the achievement target behavior includes at least one of a target number of steps and a target rehabilitation time.
(7) The information processing method according to (5) or (6),
   wherein the consecutive daily behavior includes at least one of number of consecutive days of medication and number of consecutive days of rehabilitation.
(8) The information processing method according to any one of (4) to (7),
   wherein the behavioral change includes at least one of a change in number of steps, a change in a ratio of movement means, and a change in wake-up time.
(9) The information processing method according to any one of (4) to (8),
   wherein the situation related to the difference from the reference includes at least one of a situation related to body temperature, a situation related to blood pressure, and a situation related to complexion.
(10) The information processing method according to any one of (1) to (9), further comprising:
   calculating priority of comments; and
   generating content including a comment having a priority calculated to be a high priority.
(11) The information processing method according to (10),
   wherein the priority of the comment is calculated based on a difference between a deviation value of a situation to be commented and a priority calculation reference value.
(12) The information processing method according to (11),
   wherein the priority of the comment is calculated such that the more the number of consecutive days of generation of the content including a same comment, the lower the priority of the comment.
(13) The information processing method according to (11) or (12),
   wherein, in a case where it is necessary to delve into the extracted situation based on an answer of a user to the inquiry comment, the priority of the comment is calculated such that another inquiry comment regarding the situation becomes high, and otherwise, specifically in a case where there is no need to perform delving, the priority of the comment is calculated such that the priority of the another inquiry comment becomes low.
(14) The information processing method according to any one of (11) to (13),
   wherein the comment includes a positive comment and a negative comment, regarding the extracted situation, and
   the priority of the comment is calculated such that the greater the ratio of the negative comment to the comment included in the content generated so far than the ratio of the positive comment, the higher the priority of the positive comment.
(15) The information processing method according to any one of (1) to (14),
   wherein the content includes an avatar that utters the comment.
(16) An information processing system comprising:
   an information acquisition device that acquires sensing data of a sensor provided for a user being a patient; and
   an information processing device that extracts a situation to be highlighted from daily situations of the user, the situations grasped from the sensing data acquired by the information acquisition device, and generates content including a comment on the extracted situation,
   wherein the comment includes an inquiry comment.
(17) The information processing system according to (16), further comprising
   a user terminal that presents the content generated by the information processing device to the user.
(18) An information processing device comprising:
   an extraction unit that extracts a situation to be highlighted from among daily situations of a user being a patient, the situations grasped from sensing data of a sensor provided for the user; and
   a generation unit that generates content including a comment on the situation extracted by the extraction unit,
   wherein the comment includes an inquiry comment.

### REFERENCE SIGNS LIST

1 INFORMATION ACQUISITION DEVICE
11 SENSOR
12 USER TERMINAL
2 INFORMATION PROCESSING DEVICE
21 COMMUNICATION UNIT
22 EXTRACTION UNIT
23 GENERATION UNIT
24 CALCULATION UNIT
25 STORAGE UNIT
251 PATIENT INFORMATION (LOG INFORMATION)
252 PARAMETER INFORMATION
253 COMMENT INFORMATION
254 COMMUNITY INFORMATION
255 ANONYMIZED INFORMATION
256 RECOMMENDATION INFORMATION
3 HEALTHCARE PROVIDER TERMINAL
31 COMMUNICATION UNIT
32 USER INTERFACE UNIT
4 COMMUNITY MEMBER TERMINAL
41 COMMUNICATION UNIT
42 USER INTERFACE UNIT
5 LIFE INSURANCE/HEALTH INSURANCE TERMINAL
51 COMMUNICATION UNIT
52 ANALYSIS UNIT
53 STORAGE UNIT
531 CUSTOMER/EMPLOYEE INFORMATION
6 PRODUCT/SERVICE PROVIDER TERMINAL
61 COMMUNICATION UNIT
62 USER INTERFACE UNIT
7 ANALYSIS TERMINAL
71 COMMUNICATION UNIT
72 ANALYSIS UNIT
73 USER INTERFACE UNIT

1. An information processing method to be implemented by an information processing device, the method comprising:
receiving sensing data obtained by a sensor provided for a user being a patient;
extracting a situation to be highlighted among daily situations of the user, the situations grasped from the received sensing data; and
generating content including a comment on the extracted situation,
wherein the comment includes an inquiry comment.

2. The information processing method according to claim 1,
wherein the comment includes an inquiry comment for inquiring a reason of the extracted situation.

3. The information processing method according to claim 1,
wherein the inquiry comment includes at least one of a comment related to a physical condition, a comment related to pain, a comment related to fatigue, and a comment related to mood.

4. The information processing method according to claim 1,
wherein the situation to be highlighted includes at least one of a situation related to occurrence of a specific behavior, a situation related to a behavioral change, and a situation related to a difference from a reference.

5. The information processing method according to claim 4,
wherein the specific behavior includes at least one of an achievement target behavior and a consecutive daily behavior.

6. The information processing method according to claim 5,
wherein the achievement target behavior includes at least one of a target number of steps and a target rehabilitation time.

7. The information processing method according to claim 5,
wherein the consecutive daily behavior includes at least one of number of consecutive days of medication and number of consecutive days of rehabilitation.

8. The information processing method according to claim 4,
wherein the behavioral change includes at least one of a change in number of steps, a change in a ratio of movement means, and a change in wake-up time.

9. The information processing method according to claim 4,
wherein the situation related to the difference from the reference includes at least one of a situation related to body temperature, a situation related to blood pressure, and a situation related to complexion.

10. The information processing method according to claim 1, further comprising:
calculating priority of comments; and
generating content including a comment having a priority calculated to be a high priority.

11. The information processing method according to claim 10,
wherein the priority of the comment is calculated based on a difference between a deviation value of a situation to be commented and a priority calculation reference value.

12. The information processing method according to claim 11,
wherein the priority of the comment is calculated such that the more the number of consecutive days of generation of the content including a same comment, the lower the priority of the comment.

13. The information processing method according to claim 11,
wherein, in a case where it is necessary to delve into the extracted situation based on an answer of a user to the inquiry comment, the priority of the comment is calculated such that another inquiry comment regarding the situation becomes high, and otherwise, specifically in a case where there is no need to perform delving, the priority of the comment is calculated such that the priority of the another inquiry comment becomes low.

14. The information processing method according to claim 11,
wherein the comment includes a positive comment and a negative comment, regarding the extracted situation, and
the priority of the comment is calculated such that the greater the ratio of the negative comment to the comment included in the content generated so far than the ratio of the positive comment, the higher the priority of the positive comment.

15. The information processing method according to claim 1,
wherein the content includes an avatar that utters the comment.

16. An information processing system comprising:
an information acquisition device that acquires sensing data of a sensor provided for a user being a patient; and
an information processing device that extracts a situation to be highlighted from daily situations of the user, the situations grasped from the sensing data acquired by the information acquisition device, and generates content including a comment on the extracted situation,
wherein the comment includes an inquiry comment.

17. The information processing system according to claim 16, further comprising
a user terminal that presents the content generated by the information processing device to the user.

18. An information processing device comprising:
an extraction unit that extracts a situation to be highlighted from among daily situations of a user being a patient, the situations grasped from sensing data of a sensor provided for the user; and
a generation unit that generates content including a comment on the situation extracted by the extraction unit,
wherein the comment includes an inquiry comment.
